(19) United States
(12) Patent Application Publication
(10) Pub. No.: US 2025/0134598 A1
(43) Pub. Date: May 1, 2025

(54) TRACKING SYSTEMS AND METHODS FOR IMAGE-GUIDED SURGERY

(71) Applicant: AUGMEDICS LTD., Yokneam Illit (IL)

(72) Inventors: Stuart Wolf, Yokneam (IL); Nissan Elimelech, Beerotaim (IL)

(21) Appl. No.: 18/978,615

(22) Filed: Dec. 12, 2024

Related U.S. Application Data
(63) Continuation of application No. 18/631,877, filed on Apr. 10, 2024, now Pat. No. 12,201,384.
Application No. 18/631,877 is a continuation of application No. 18/470,809, filed on Sep. 20, 2023, now Pat. No. 11,980,429.
Application No. 18/470,809 is a continuation of application No. 16/200,144, filed on Nov. 26, 2018, now Pat. No. 11,766,296.

(51) Int. Cl. A61B 34/20; A61B 34/00; A61B 34/10; A61B 90/00; G02B 27/00; G02B 27/01

(52) U.S. Cl. CPC A61B 34/20; A61B 34/25; A61B 90/36; G02B 27/0093; G02B 27/0172; A61B 34/10; A61B 2034/2055; A61B 2090/365; G02B 2027/0178

(57) ABSTRACT

Apparatus and methods are described including tracking a tool portion and a patient marker from a first line of sight, using a first tracking device disposed upon a first head-mounted device that includes a display. The tool portion and the patient marker are tracked from a second line of sight, using a second tracking device. When a portion of the patient marker and the tool portion are both within the first line of sight, an augmented reality image is generated upon the first display based upon data received from the first tracking device and without using data from the second tracking device. When at least the patient marker portion and the tool portion are not both within the first line of sight, a virtual image of the tool and anatomy of the patient is generated using data 10 received from the second tracking device. Other applications are also described.

### CROSS-REFERENCE TO RELATED APPLICATIONS

This application is a continuation of U.S. patent application Ser. No. 18/631,877, filed Apr. 10, 2024, which is a continuation of U.S. patent application Ser. No. 18/470,809, filed Sep. 20, 2023, which is a continuation of U.S. patent application Ser. No. 16/200,144, filed Nov. 26, 2018. Each of the foregoing applications is hereby incorporated by reference herein in its entirety.

### FIELD OF EMBODIMENTS OF THE INVENTION

The present invention relates to generally to an augmented reality system, and specifically to a tracking system for an augmented reality system that is used to perform image-guided surgery.

### BACKGROUND

A head-mounted display is sometimes used as part of an augmented reality system. The display is used to generate an augmented reality scene, in which a scene that is being viewed by a user of the head-mounted display is altered, typically by being augmented or supplemented. The alteration is computer generated, and typically involves presenting real-time video, and/or non-real-time images, to the user while the user is gazing at the scene.

In some cases, an augmented reality system is used for performing image-guided surgery, as part of a medical procedure. For example, a computer-generated image may be presented to a healthcare professional who is performing the procedure. The image may be presented on a head-mounted display such that the image is aligned with an anatomical portion of a patient who is undergoing the procedure. Although some misalignment of the image with the patient's body may be acceptable, for satisfactory presentation of the images the misalignment may typically not be more than about 2-3 mm. In order to account for such a limit on the misalignment of the patient's anatomy with the presented images, the position of the patient's body or a portion thereof is typically tracked.

In some cases, an image of a tool that is used to perform the procedure is incorporated into the image that is displayed on the head-mounted display. In order to incorporate an image of the tool into the image, in a manner in which the position of the tool with respect to the image and/or the patient's anatomy is accurately reflected, the position of the tool or a portion thereof is typically tracked.

Triangulation techniques are commonly employed for tracking positions of a patient's body and/or a tool. In such techniques, a plurality of imaging devices, which are disposed at known locations with respect to each other, are used to detect a feature (such as a marker) on the patient's body, and/or on the tool. The location of the feature is then derived, using a combination of the known locations of the imaging devices, as well as the location of the feature as detected by each of the imaging devices.

### SUMMARY OF EMBODIMENTS

In accordance with some applications of the present invention, a first healthcare professional (e.g., a surgeon performing a procedure) wears a first head-mounted device. Typically, the head-mounted device includes one or more head-mounted displays. For some applications, the head-mounted displays are generally similar to those described in U.S. Pat. No. 9,928,629 to Benishti, which is incorporated herein by reference. For example, the head-mounted displays may include a combiner that is controlled by a computer processor, such as to display an augmented reality image to the healthcare professional. For some applications, the image is presented on the head-mounted display such that (a) a computer-generated image is projected onto a first portion of the display, and (b) the computer-generated image is aligned with an anatomical portion of a patient who is undergoing the procedure, with the anatomical portion of a patient visible through a second portion of the display. Typically, the computer-generated image includes a virtual image of the tool overlaid upon a virtual image of the patient's anatomy. For some applications, a portion of the tool that would not otherwise be visible to the healthcare professional (for example, by virtue of being hidden by the patient's anatomy) is included in the computer-generated image.

Typically, the head-mounted device includes a tracking device that is configured to facilitate determination of the location and orientation of the head-mounted device with respect to a portion of the patient's body (e.g., the patient's back), and/or the position and orientation of the tool with respect to the patient's body. For example, the tracking device may include an image-capturing device, such as a camera, that is configured to image a patient marker and/or a tool marker. Typically, the patient marker is configured to provide data that is sufficient for the computer processor to determine the location and orientation of the head-mounted device with respect to the portion of the patient's body using data collected from a single tracking device that is disposed on the head-mounted display. For example, the patient marker may include an array of elements that is visible to the tracking device of the head-mounted device, and that is configured such that at any location and orientation of the head-mounted device with respect to the patient marker, the array of elements has an appearance that is unique to that location and orientation. In this manner, the computer processor is able to determine the location and orientation of the head-mounted device with respect to the portion of the patient's body without requiring the use of triangulation techniques. Typically, a single camera is used in the tracking device of the head-mounted device. For some applications, the camera is a high-speed camera. For example, the camera may acquire more than 50 frames per second.

Typically, in order to generate the augmented reality image upon the head-mounted display, a computer processor determines the location and orientation of the head-mounted device with respect to a portion of the patient's body (e.g., the patient's back), and/or the position and orientation of the tool with respect to the portion of the patient's body. As described hereinabove, in general, the patient marker is configured to provide data that is sufficient for the computer processor to determine the location and orientation of the head-mounted device with respect to the portion of the patient's body using data collected from a single tracking device that is disposed on the head-mounted device. However, for some applications, at least under certain conditions, the computer processor is configured to incorporate tracking data that is received from at least one additional tracking device (i.e., a tracking device in addition to the tracking device that is included in the head-mounted device of the first healthcare professional), in order to generate the image upon the head-mounted display of the first healthcare professional.

For some such applications, the computer processor is configured to incorporate the additional data in cases in which the first tracking device that is included in the head-mounted device of the first healthcare professional loses its line of sight with the patient marker and/or the tool marker and/or portions thereof. For example, the computer processor may be configured to receive data from a tracking device of an additional head-mounted device that is configured to be worn by an additional healthcare professional who is present in the procedure (e.g., an accompanying surgeon or a nurse). Typically, the additional head-mounted device is generally similar to the first head-mounted device, and the tracking device of the additional head-mounted device is generally similar to that of the first head-mounted device. For some applications, when at least a portion of the patient marker and a portion of the tool (e.g., the tool marker) are both within the line of sight of the first tracking device, the computer processor generates an augmented reality image upon the head-mounted display, based upon data received from first tracking device and without using data received from the additional tracking device. When at least the portion of the patient marker and the portion of the tool are not both within the line of sight of the first tracking device, the computer processor generates an augmented reality image upon the first head-mounted display, at least partially based upon data received from the additional tracking device.

There is therefore provided, in accordance with some applications of the present invention, a method for use with a tool configured to be placed within a portion of a body of a patient, the method including:
tracking at least a portion of the tool and a patient marker that is placed upon the patient's body from a first line of sight, using a first tracking device that is disposed upon a first head-mounted device that is worn by a first person, the first head-mounted device including a first head-mounted display;
tracking at least the portion of the tool and the patient marker, from a second line of sight, using a second tracking device; and
using at least one computer processor:
   when at least a portion of the patient marker and the portion of the tool are both within the first line of sight, generating an augmented reality image upon the first head- mounted display based upon data received from the first tracking device and without using data from the second tracking device, the augmented reality image including (a) a virtual image of the tool and anatomy of the patient, overlaid upon (b) the patient's body; and
   when at least the portion of the patient marker and the portion of the tool are not both within the first line of sight, generating a virtual image of the tool and anatomy of the patient upon the first head-mounted display, at least partially based upon data received from the second tracking device.

In some applications, tracking the portion of the tool includes tracking a tool marker. In some applications, tracking at least the portion of the tool and the patient marker, from the second line of sight, using the second tracking device, includes tracking at least the portion of the tool and the patient marker from the second line of sight, using a second tracking device that is disposed in a stationary position. In some applications, tracking at least the portion of the tool and the patient marker using the first tracking device includes tracking at least the portion of the tool and the patient marker using a first camera, and tracking at least the portion of the tool and the patient marker using the second tracking device includes tracking at least the portion of the tool and the patient marker using a second camera.

In some applications, generating the virtual image of the tool and anatomy of the patient upon the first head-mounted display, at least partially based upon data received from the second tracking device includes:
in response to the portion of the patient marker being within the first line of sight, and the portion of the tool not being within the first line of sight:
   determining a position of the tool with respect to the subject's anatomy using data received from the second tracking device;
   generating the virtual image of the tool and anatomy of the patient upon the first head-mounted display, based upon the determined position of the tool with respect to the subject's anatomy;
   determining a position of the patient's body with respect to the first head-mounted device based upon data received from the first tracking device; and
   overlaying the virtual image upon the patient's body, based upon the determined position of the patient's body with respect to the first head-mounted device.

In some applications, generating the virtual image of the tool and anatomy of the patient upon the first head-mounted display, at least partially based upon data received from the second tracking device includes:
in response to the portion of the tool being within the first line of sight, and the portion of the patient marker not being within the first line of sight:
   determining a position of the tool with respect to the subject's anatomy using data received from the second tracking device;
   generating the virtual image of the tool and anatomy of the patient upon the first head-mounted display, based upon the determined position of the tool with respect to the subject's anatomy.

In some applications, generating the virtual image of the tool and anatomy of the patient upon the first head-mounted display further includes overlaying the virtual image upon the patient's body, based upon a position of the patient's body with respect to the first head-mounted device as determined based upon data received from the first tracking device at a time when the portion of the patient marker was within the first line of sight.

In some applications, overlaying the virtual image upon the patient's body includes tracking movements of the head-mounted device between the time when the portion of the patient marker was within the first line of sight and the portion of the patient marker not being within the first line of sight, using an inertial-measurement unit disposed upon the first head-mounted device.

In some applications, generating the virtual image of the tool and anatomy of the patient upon the first head-mounted display, at least partially based upon data received from the second tracking device includes:
in response to the portion of the tool and the portion of the patient marker both not being within the first line of sight:
   determining a position of the tool with respect to the subject's anatomy using data received from the second tracking device;
   generating the virtual image of the tool and anatomy of the patient upon the first head-mounted display, based upon the determined position of the tool with respect to the subject's anatomy.

In some applications, generating the virtual image of the tool and anatomy of the patient upon the first head-mounted display further includes overlaying the virtual image upon the patient's body, based upon a position of the patient's body with respect to the first head-mounted device as determined based upon data received from the first tracking device at a time when the portion of the patient marker was within the first line of sight. In some applications, overlaying the virtual image upon the patient's body includes tracking movements of the head-mounted device between the time when the portion of the patient marker was within the first line of sight and the portion of the patient marker not being within the first line of sight, using an inertial-measurement unit disposed upon the first head-mounted device.

In some applications, tracking at least the portion of the tool and the patient marker, from the second line of sight, using the second tracking device, includes tracking at least the portion of the tool and the patient marker from the second line of sight, using a second tracking device that is disposed upon a second head-mounted device that is worn by a second person. In some applications, the second head-mounted device includes a second head-mounted display, the method further including generating a further augmented-reality image upon the second head-mounted display.

There is further provided, in accordance with some applications of the present invention, apparatus for use with a tool configured to be placed within a portion of a body of a patient, the apparatus including:
a patient marker configured to be placed upon the patient's body;
a first head-mounted device including a first head-mounted display, and a first tracking device that is configured to track at least a portion of the tool and the patient marker from a first line of sight;
a second tracking device that is configured to track at least the portion of the tool and the patient marker from a second line of sight; and
at least one computer processor configured:
   when at least a portion of the patient marker and the portion of the tool are both within the first line of sight, to generate an augmented reality image upon the first head-mounted display, based upon data received from the first tracking device and without using data from the second tracking device, the augmented reality image including (a) a virtual image of the tool and anatomy of the patient, overlaid upon (b) the patient's body; and
   when at least the portion of the patient marker and the portion of the tool are not both within the first line of sight, to generate a virtual image of the tool and anatomy of the patient upon the first head-mounted display, at least partially based upon data received from the second tracking device.

There is further provided, in accordance with some applications of the present invention, a method for use with a tool configured to be placed within a portion of a body of a patient, the method including:
tracking at least a portion of the tool and a patient marker that is placed upon the patient's body from a first line of sight, using a first tracking device that is disposed upon a first head-mounted device that is worn by a first person, the first head-mounted device including a first head-mounted display;
tracking at least the portion of the tool and the patient marker from a second line of sight, using a second tracking device that is disposed upon a second head-mounted device that is worn by a second person; and
using at least one computer processor, generating an augmented reality image upon the first head-mounted display, based upon data received from the first tracking device in combination with data received from the second tracking device, the augmented reality image including (a) a virtual image of the tool and anatomy of the patient, overlaid upon (b) the patient's body.

In some applications, tracking the portion of the tool includes tracking a tool marker. In some applications, the second head-mounted device includes a second head-mounted display, the method further including generating a further augmented-reality image upon the second head-mounted display. In some applications, tracking at least the portion of the tool and the patient marker using the first tracking device includes tracking at least the portion of the tool and the patient marker using a first camera, and tracking at least the portion of the tool and the patient marker using the second tracking device includes tracking at least the portion of the tool and the patient marker using a second camera.

In some applications, generating the augmented reality image upon the first head-mounted display includes:
determining a position of the tool with respect to the subject's anatomy using data received from the first tracking device in combination with data received from the second tracking device;
generating the virtual image of the tool and anatomy of the patient upon the first head-mounted display, based upon the determined position of the tool with respect to the subject's anatomy;
determining a position of the patient's body with respect to the first head-mounted device based upon data received from the first tracking device; and
overlaying the virtual image upon the patient's body, based upon the determined position of the patient's body with respect to the first head-mounted device.

There is further provided, in accordance with some applications of the present invention, apparatus for use with a tool configured to be placed within a portion of a body of a patient, the apparatus including:
a patient marker configured to be placed upon the patient's body;
a first head-mounted device configured to be worn by a first person, the first head-mounted device including a first head-mounted display, and a first tracking device that is configured to track at least a portion of the tool and the patient marker from a first line of sight;
a second head-mounted device configured to be worn by a second person, the second head-mounted device including a second tracking device that is configured to track at least a portion of the tool and the patient marker from a second line of sight; and
at least one computer processor configured to generate an augmented reality image upon the first head-mounted display, based upon data received from the first tracking device in combination with data received from the second tracking device, the augmented reality image including (a) a virtual image of the tool and anatomy of the patient, overlaid upon (b) the patient's body.

The present invention will be more fully understood from the following detailed description of applications thereof. taken together with the drawings, in which:

### BRIEF DESCRIPTION OF THE DRAWINGS

FIG. 1 is a schematic illustration of image-guided surgery being performed upon a patient, in accordance with some applications of the present invention;

FIG. 2 is a schematic illustration of a head-mounted device, in accordance with some applications of the present invention;

FIGS. 3A and 3B are schematic illustrations of examples of displays of head-mounted devices as worn by respective healthcare professionals, in accordance with some applications of the present invention;

FIGS. 4A and 4B are schematic illustrations of examples of displays of head-mounted devices as worn by respective healthcare professionals, when the line of sight between a tracking device of one of the healthcare professionals with respect to a patient marker is at least partially blocked, in accordance with some applications of the present invention;

FIGS. 5A and 5B are schematic illustrations of examples of displays of head-mounted devices as worn by respective healthcare professionals, when the line of sight between a tracking device of one of the healthcare professionals with respect to a patient marker is at least partially blocked, in accordance with some applications of the present invention; and

FIGS. 6A and 6B are schematic illustration of examples of displays of head-mounted devices as worn by respective healthcare professionals, when the line of sight between a tracking device of one of the healthcare professionals with respect to a patient marker is at least partially blocked, in accordance with some applications of the present invention.

### DETAILED DESCRIPTION OF EMBODIMENTS

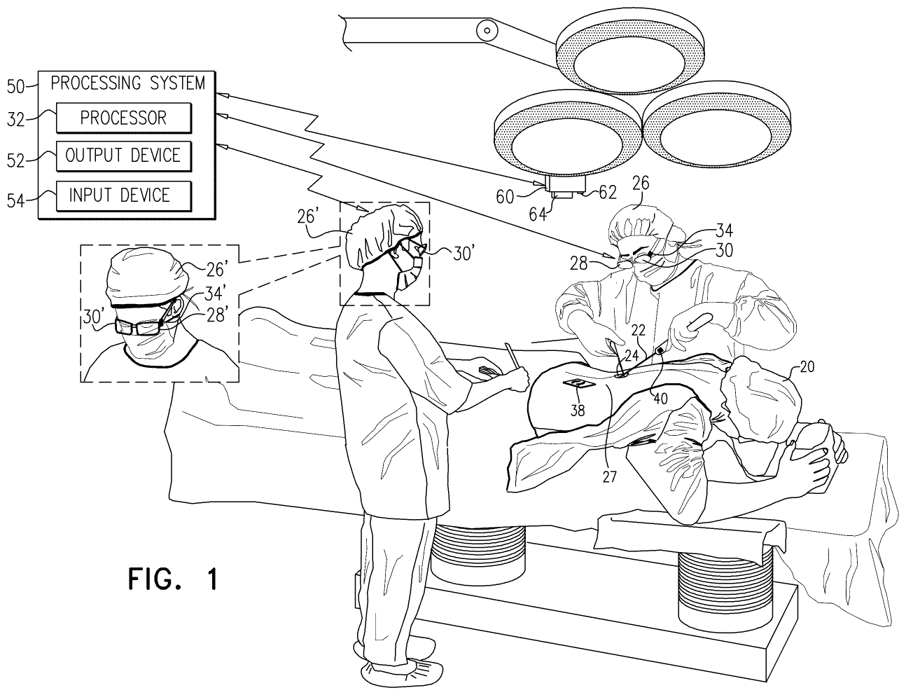

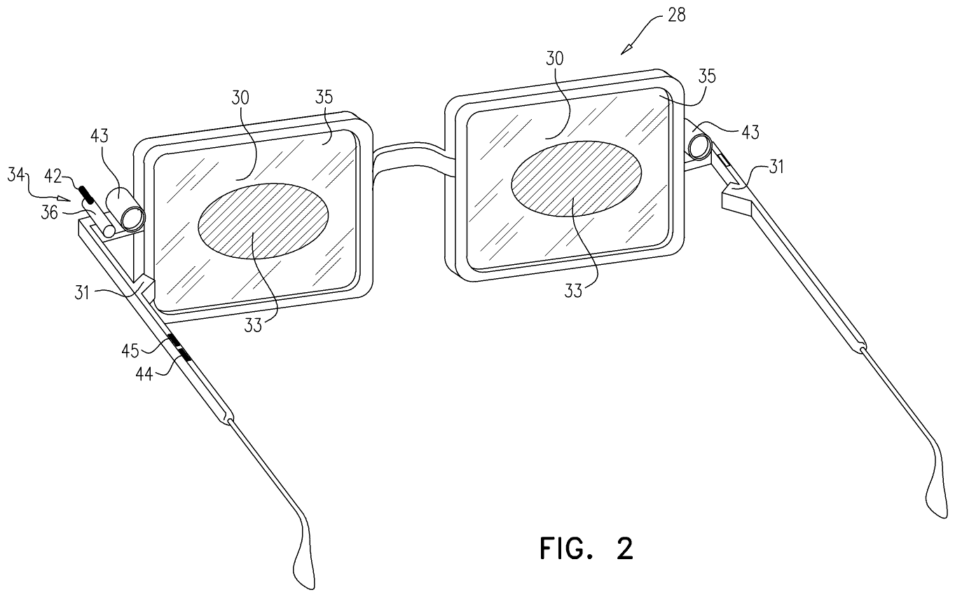

Reference is now made to FIG. 1, which is a schematic illustration of a medical procedure that incorporates image-guided surgery being performed upon a patient 20, in accordance with some applications of the present invention. In the medical procedure shown in FIG. 1, a tool 22 is used to perform an action with respect to the patient's back, the tool being inserted via an incision 24 on the patient's back 27. However, the apparatus and techniques described herein may be used in any surgical procedure that is performed upon a patient's body, mutatis mutandis. Reference is also made to FIG. 2, which is a schematic illustration of a head-mounted device 28, in accordance with some applications of the present invention.

For some applications, a first healthcare professional 26 (e.g., a surgeon performing the procedure) wears a first head-mounted device 28. Typically, the head-mounted device includes one or more head-mounted displays 30. For some applications, the head-mounted displays are generally similar to those described in U.S. Pat. No. 9,928,629 to Benishti, which is incorporated herein by reference. For example, the head-mounted displays may include a combiner that is controlled by a computer processor (e.g., computer processor 32 and/or computer processor 45 described hereinbelow), such as to display an augmented reality image to the healthcare professional. For some applications, the image is presented on head-mounted display 30 such that (a) a computer-generated image is projected onto a first portion 33 of the display by projector 31, and (b) the computer-generated image is aligned with an anatomical portion of a patient who is undergoing the procedure, with the anatomical portion of a patient being visible through a second portion 35 of the display. Typically, the computer-generated image includes a virtual image of the tool overlaid upon a virtual image of the patient's anatomy. For some applications, a portion of the tool that would not otherwise be visible to the healthcare professional (for example, by virtue of being hidden by the patient's anatomy) is included in the computer-generated image.

Although some misalignment of the image with the patient's body may be acceptable, for satisfactory presentation of the images the misalignment may typically not be more than about 2-3 mm. In order to account for such a limit on the misalignment of the patient's anatomy with the presented images, the position of the patient's body, or a portion thereof, with respect to the head-mounted device is typically tracked. In some cases, an image of a tool that is used to perform the procedure is incorporated into the image that is displayed on the head-mounted display. In order to incorporate an image of the tool into the image, in a manner in which the position of the tool with respect to the patient's anatomy is accurately reflected, the position of the tool or a portion thereof (e.g., the tool marker) is typically tracked. It is typically desirable to determine the location of the tool with respect to the patient's body such that errors in the determined location of the tool with respect to the patient's body are less than 2 mm.

Typically, head-mounted device 28 includes a tracking device 34 that is configured to facilitate determination of the location and orientation of head-mounted device 28 with respect to a portion of the patient's body (e.g., the patient's back) and/or with respect to tool 22, and/or the position and orientation of the tool with respect to the portion of the patient's body. For example, the tracking device may include an image-capturing device 36, such as a camera, that is configured to image a patient marker 38 and/or a tool marker 40. Typically, a single camera is used in the tracking device of the head-mounted device. For some applications, the camera is a high-speed camera. For example, the camera may acquire more than 50 frames per second.

For some applications, tracking device 34 includes a light source 42, which is mounted on the head-mounted device. The light source is typically configured to irradiate the patient marker and/or the tool marker, such that light reflects from the markers toward the camera. For some applications, image-capturing device 36 is a monochrome camera that includes a filter that is configured to only allow light to pass through that is of a similar wavelength to the light that is generated by the light source. For example, the light source may be an infrared light source (for example, a light source that generates light at a wavelength of between 700 nm and 1000 nm (e.g., between 700 nm and 800 nm)), and the camera may include a corresponding infrared filter. For some applications, an inertial-measurement unit 44 (e.g., an inertial-measurement unit configured to measure in 6 degrees-of-freedom) is disposed on the head-mounted device, as described in further detail hereinbelow. For some applications, the head-mounted device includes additional cameras 43, which are configured to capture images of scenes in the visible spectrum, as described in U.S. Pat. No. 9,928,629 to Benishti, which is incorporated herein by reference. For some applications, head-mounted device 28 includes additional components, for example, as described in U.S. Pat. No. 9,928,629 to Benishti, which is incorporated herein by reference.

Typically, in order to generate an augmented reality image upon display 30, a computer processor determines the location and orientation of head-mounted device 28 with respect to a portion of the patient's body (e.g., the patient's back), and/or the position and orientation of the tool with respect to the patient's body. For example, a computer processor 45 that is integrated within the head-mounted device may perform the aforementioned functionalities. Alternatively or additionally, computer processor 32, which is disposed externally to the head-mounted device and is typically in wireless communication with the head-mounted device may be used to perform these functionalities. Computer processor 32 typically comprises a portion of a processing system 50 that is used with the head-mounted device in order to facilitate the image-guided surgery. For some applications, the processing system additionally includes an output device 52 (e.g., a display, such as a monitor) for outputting information to an operator of the system, and/or an input device 54 (such as a pointing device, a keyboard, a mouse, etc.) configured to allow the operator to input data into the system. In general, in the context of the present application, when a computer processor is described as performing certain steps, these steps may be performed by external computer processor 32, and/or computer processor 45 that is integrated within the head-mounted device.

For some applications, the patient marker and/or the tool marker includes reflective elements that are configured to reflect light that is generated by light source 42. For some such applications, the location and orientation of a portion of the subject's body (e.g., the subject's back) with respect to the head-mounted device is tracked, by directing light from light source 42 toward a region of interest in which the patient marker is disposed. Alternatively or additionally, the location and orientation of the tool with respect to the portion of the subject's body, is tracked by directing light from light source 42 toward a region of interest in which the patient marker and/or the tool marker is disposed. Typically, image-capturing device 36 is disposed upon the head-mounted device in close proximity to the light source, such that the image-capturing device is configured to capture light that is retro-reflected from the patient marker and/or the tool marker.

As described hereinabove, for some applications, the image-capturing device is a monochrome camera that includes a filter that is configured to only allow light to pass through that is of a similar wavelength to the light that is generated by the light source. For such applications, the camera typically receives a grayscale image showing the reflective elements of the tool marker and/or the patient marker. Typically, the computer processor determines the location of a portion of the subject's body (e.g., the subject's back) with respect to the head-mounted device by analyzing the images acquired by the image-capturing device. Further typically, the computer processor determines the location and orientation of the tool with respect to the portion of the subject's body, by analyzing the images acquired by the image-capturing device.

It is noted that the above-described technique for tracking the patient marker and/or the tool marker is presented by way of example, and that for some applications, alternative techniques are used for tracking the patient marker and/or the tool marker. For example, the patient marker and/or the tool marker may include light-absorbing elements, and/or light-generating elements, and the image-capturing device may be configured to track the patient marker and/or the tool marker by detecting these elements. Alternatively or additionally, a different type of detector may be used for tracking the patient marker and/or the tool marker.

Typically, the patient marker is configured to provide data that is sufficient for the computer processor to determine the location and orientation of the head-mounted device with respect to the portion of the patient's body using data collected from a single tracking device that is disposed on the head-mounted display. For example, the patient marker may include an array of elements that is visible to the tracking device of the head-mounted device, and that is configured such that at any location and orientation of the head-mounted device with respect to the patient marker, the array of elements has an appearance that is unique to that location and orientation. In this manner, the computer processor is able to determine the location and orientation of the head-mounted device with respect to the portion of the patient's body without requiring the use of triangulation techniques.

As described in the above paragraph, in general, the patient marker is configured to provide data that is sufficient for the computer processor to determine the location and orientation of the head-mounted device with respect to the portion of the patient's body using data collected from a single tracking device that is disposed on the head-mounted display. However, for some applications, at least under certain circumstances, the computer processor is configured to incorporate tracking data that is received from an additional tracking device (i.e., an additional tracking device to first tracking device 34), in order to generate the image upon head-mounted display 30 of first head-mounted device 28 of first healthcare professional 26.

For some such applications, the computer processor is configured to incorporate the additional data in cases in which tracking device 34 loses its line of sight with the patient marker and/or the tool marker and/or portions thereof. An example of this is shown in FIG. 1, which shows that the right hand of first healthcare professional 26 is blocking the line of sight of his/her tracking device 34 with respect to patient marker 38. For some applications, in such cases, the computer processor is configured to receive data from a tracking device 34′ of an additional head-mounted device 28′ that is configured to be worn by an additional healthcare professional 26′ who is present in the procedure (e.g., an accompanying surgeon, or a nurse), e.g., as shown in FIG. 1. Typically, the additional head-mounted device 28′ is generally similar to the first head-mounted device 28, and the tracking device 34′ of the additional head-mounted device is generally similar to that of the first head-mounted device. For some applications, when at least a portion of the patient marker and a portion of the tool (e.g., the tool marker) are both within the line of sight of the first tracking device 34, the computer processor generates an augmented reality image upon the head-mounted display 30, based upon data received from first tracking device 34 and without using data received from tracking device 34′. When at least the portion of the patient marker and the portion of the tool are not both within the line of sight of first tracking device 34, the computer processor generates an augmented reality image upon head-mounted display 30, at least partially based upon data received from second tracking device 34′.

Alternatively or additionally, a tracking device 60, which is not mounted on a head-mounted device, is disposed in the operating room. Typically, tracking device 60 is disposed in a stationary position within the operating room. For example, tracking device 60 may be ceiling-mounted, wall-mounted, and/or disposed on a stand, such as a tripod. For some applications, tracking device 60 includes a light source 62 and an image-capturing device 64, which function in a generally similar manner to that described hereinabove with reference to light source 42 and image-capturing device 36.

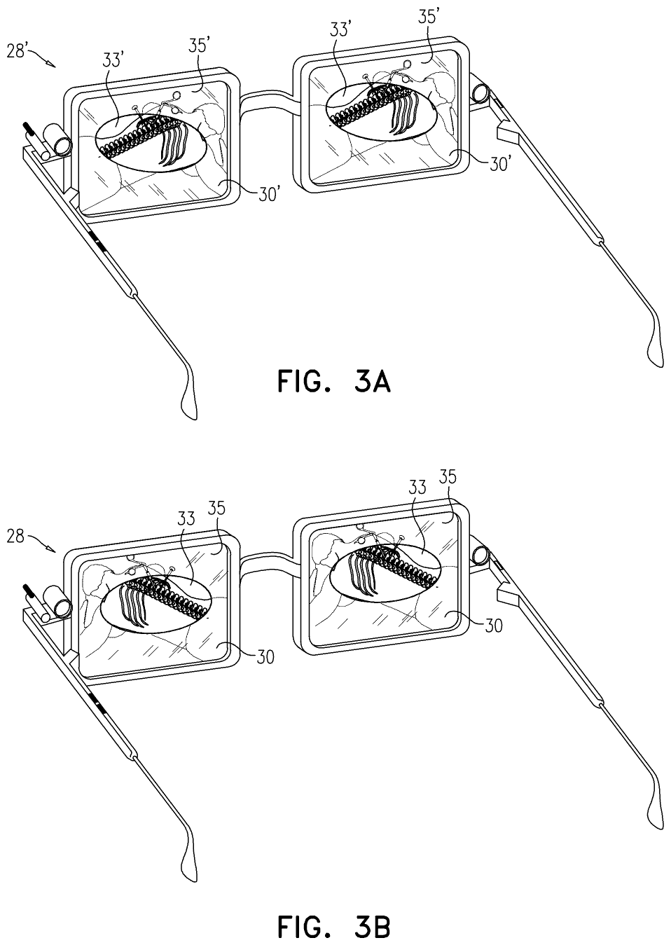

Reference is now made to FIGS. 3A and 3B, which are schematic illustration of examples of displays 30′, 30 of head-mounted devices 28′, 28 as worn by respective healthcare professionals 26′, 26, in accordance with some applications of the present invention. FIG. 3A shows an example of displays 30′ of second healthcare professional 26′, who is shown on the right side of the patient in FIG. 1, and FIG. 3B shows an example of displays 30 of first healthcare professional 26, who is shown on the left side of the patient in FIG. 1. Typically, the image that is generated upon each of head-mounted displays 30 and head-mounted displays 30′ is an augmented-reality view showing virtual patient anatomy aligned with the actual patient anatomy and a virtual tool aligned with the virtual anatomy. As described hereinabove, for some applications, the virtual tool and virtual anatomy are displayed upon a first portion 33 of head-mounted display 30, 30′, and the actual patient anatomy is visible through a second portion 35 of head-mounted display 30, 30′. For some applications, the computer processor is configured to generate such a view both in 2D and 3D. In order to generate such a view, it is typically necessary to track the location and orientation of the head-mounted device relative to the patient, in order to correctly align the virtual anatomy with the actual patient anatomy. FIGS. 3A and 3B show how the respective head-mounted displays typically appear, when tracking devices 34 of each of the healthcare professionals has a clear line of sight of the patient marker.

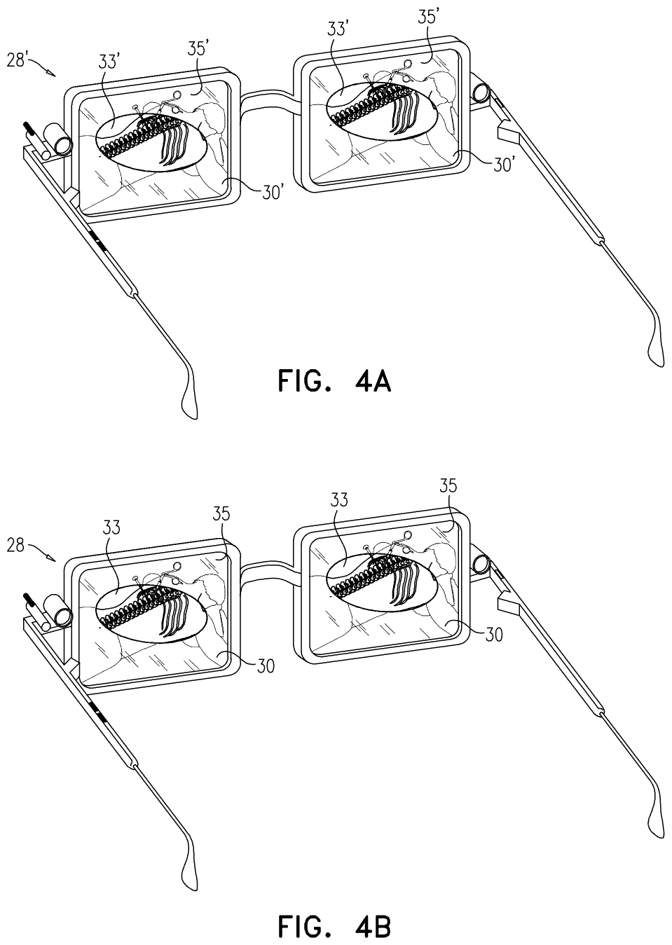

Reference is now made to FIGS. 4A and 4B, which are schematic illustration of examples of displays 30′, 30 of head-mounted devices 28′, 28 as worn by respective healthcare professionals 26′, 26, when the line of sight between tracking device 34 of first healthcare professional 26 with respect to patient marker 38 is at least partially blocked, in accordance with some applications of the present invention. FIG. 4A shows an example of display 30′ of second healthcare professional 26′, who is shown on the right side of the patient in FIG. 1, and FIG. 4B shows an example of display 30 of first healthcare professional 26, who is shown on the left side of the patient in FIG. 1.

For some such applications, the computer processor generates a virtual image upon head-mounted display 30 of first healthcare professional 26 that shows the virtual view of the second healthcare professional 26′ (i.e., the second healthcare professional's view of the virtual anatomy and the virtual tool), as determined based upon the data received from second tracking device 34′.

For example, the overall view of the second healthcare professional (including both his/her view of the virtual anatomy and the virtual tool, as well as his/her view of the actual patient anatomy) may be displayed upon head-mounted display 30 of the first healthcare professional. Such an example is shown in FIGS. 4A and 4B, which show the head-mounted displays 30 of first healthcare professional 26 showing the same overall view as that of the second healthcare professional 26′.

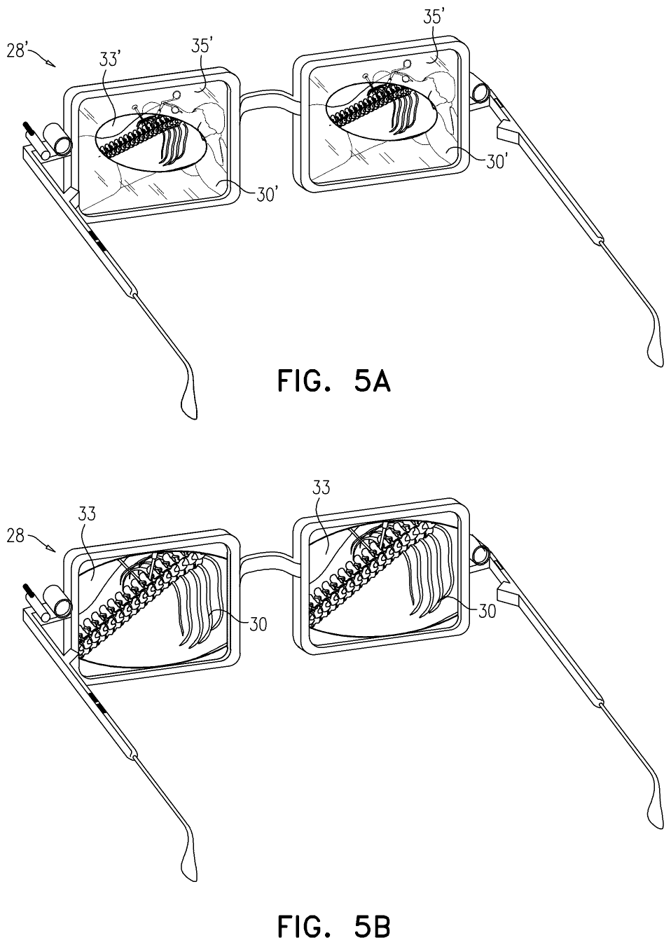

Reference is now made to FIGS. 5A and 5B, which are schematic illustration of examples of displays 30′, 30 of head-mounted devices 28′, 28 as worn by respective healthcare professionals 26′, 26, when the line of sight between tracking device 34 of first healthcare professional 26 with respect to patient marker 38 is at least partially blocked, in accordance with some applications of the present invention. FIG. 5A shows an example of display 30′ of second healthcare professional 26′, who is shown on the right side of the patient in FIG. 1, and FIG. 5B shows an example of display 30 of first healthcare professional 26, who is shown on the left side of the patient in FIG. 1. For some applications, when the line of sight between tracking device 34 of first healthcare professional 26 with respect to patient marker 38 is at least partially blocked, the virtual image (of the tool and the anatomy) from the line of sight of the second healthcare professional is displayed, such that it fills substantially the whole head-mounted display 30 of the first healthcare professional, and the first healthcare professional is not be shown any of the actual patient anatomy via a transparent portion of the display. An example of such an embodiment is shown in FIGS. 5A and 5B, which show the virtual image from head-mounted displays 30′ (shown in FIG. 5A) displayed within portion 33 of the head-mounted displays 30 of the first healthcare professional, and portion 33 filling substantially the whole of head-mounted displays 30 of the first healthcare professional (shown in FIG. 5B).

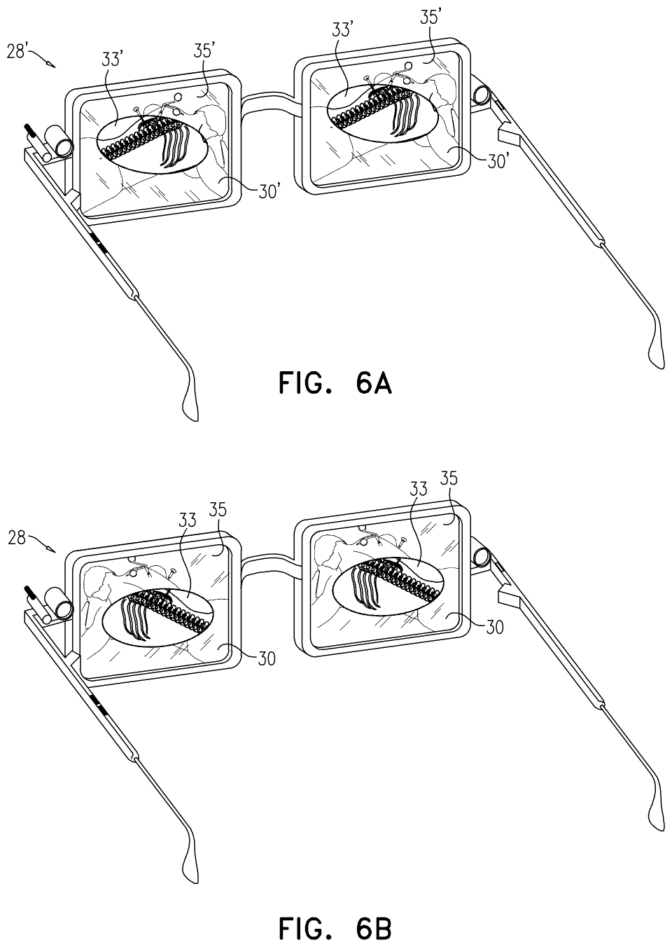

Reference is now made to FIGS. 6A and 6B, which are schematic illustration of examples of displays 30′, 30 of head-mounted devices 28′, 28 as worn by respective healthcare professionals 26′, 26, when the line of sight between tracking device 34 of first healthcare professional 26 with respect to patient marker 38 is at least partially blocked, in accordance with some applications of the present invention. FIG. 6A shows an example of display 30′ of second healthcare professional 26′, who is shown on the right side of the patient in FIG. 1, and FIG. 6B shows an example of display 30 of first healthcare professional 26, who is shown on the left side of the patient in FIG. 1. For some applications, in response to detecting that tracking device 34 has lost its line of sight of the patient marker, such that the location and/or orientation of the head-mounted device relative to the patient cannot be determined to a given level of accuracy using tracking device 34, the computer processor generates an image of the virtual tool within the virtual anatomy of the subject, but without regard to aligning the computer-generated image with the actual patient anatomy. For some such applications, the virtual image that is generated in portion 33 of display 30 of the first healthcare professional continues to be shown from the first healthcare professional's previous known line of sight, but the position of the tool with respect to the anatomy is updated based upon data received from tracking device 34′. Second portion 35 of the display of the first healthcare professional is kept transparent such that the first healthcare professional sees the patient's anatomy from his/her own current line of sight. An example of such an embodiment is shown in FIGS. 6A and 6B. As shown in FIG. 6B, since changes in the location of head-mounted device 28 with respect to the patient marker are not tracked and accounted for, this may result in a slight misalignment of the virtual image (shown in portion 33) with respect to the patient's body (shown in portion 35). In this regard, it is noted that, in general, the first healthcare professional uses the virtual image of the tool overlaid upon the virtual image of the patient's anatomy, for navigation of the tool. As such, the healthcare professional is typically able to continue to navigate the tool, even though the virtual image of the tool and the patient's anatomy is not aligned with his/her view of the patient's anatomy.

For some applications, generally similar techniques to those described in the above paragraph are performed, but with the additional tracking data that is used for generating an image on head-mounted display 30 being received from tracking device 60, as an alternative to, or in addition to, being received from tracking device 34′ of second head-mounted device 28′.

For some applications, in response to detecting that tracking device 34 has lost its line of sight of the tool marker, such that the location and/or orientation of the tool with respect to the patient cannot be determined to a given level of accuracy, the computer processor determines the location of the tool relative to the patient, using data received from tracking device 34′ and/or tracking device 60. Typically, a virtual image, which includes the virtual patient anatomy and the virtual tool shown at its current location, is displayed on head-mounted display 30′ of head- mounted device 28, with the current location of the tool with respect to the patient having been determined based upon the data received from tracking device 34′ and/or tracking device 60.

For some applications, the computer processor is configured to incorporate tracking data that is received from an additional tracking device (i.e., a tracking device in addition to tracking device 34) in order to generate an image upon head-mounted display 30 of first head-mounted device 28, even when the patient marker and the tool marker are within the line of sight of tracking device 34. For some applications, the computer processor determines the location of the tool with respect to the patient, using a combination of data received from tracking device 34′ and data received from tracking device 34, and/or using a combination of data received from tracking device 60 and data received from tracking device 34. For example, the computer processor may determine an average (e.g., a mean) current location of the tool with respect to the patient, using the aforementioned combinations of received data, and the computer processor may the generate an image of a virtual tool on virtual anatomy upon head-mounted display 30, in which the tool is positioned at the determined current position.

For some applications, even if a portion of the tracking elements on the patient marker become obscured such that they are not within the line of sight of tracking device 34, the computer processor continue to track the location of the head-mounted device with respect to the patient by tracking the marker using a tracking algorithm (e.g., using a Kalman filter). Typically, in such cases, at least while the patient marker is partially obscured, the computer processor does not continue to actively identify the marker. Rather, the computer processor continues to track the already-identified marker using the aforementioned tracking algorithm. For some applications, if the patient marker becomes obscured (e.g., partially obscured or fully obscured) such that at least a portion of the patient marker is not within the line of sight of tracking device 34, the computer processor continues to determine the location of the patient relative to the head-mounted device, using inertial measurement unit 44, in combination with the last location of the patient marker as determined using data from tracking device 34.

Applications of the invention described herein can take the form of a computer program product accessible from a computer-usable or computer-readable medium (e.g., a non-transitory computer-readable medium) providing program code for use by or in connection with a computer or any instruction execution system, such as computer processor 32 and/or 45. For the purpose of this description, a computer-usable or computer readable medium can be any apparatus that can comprise, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Typically, the computer-usable or computer readable medium is a non-transitory computer-usable or computer readable medium.

Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD.

A data processing system suitable for storing and/or executing program code will include at least one processor (e.g., computer processor 32 and/or 45) coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution. The system can read the inventive instructions on the program storage devices and follow these instructions to execute the methodology of the embodiments of the invention.

Network adapters may be coupled to the processor to enable the processor to become coupled to other processors or remote printers or storage devices through intervening private or public networks. Modems, cable modem and Ethernet cards are just a few of the currently available types of network adapters.

Computer program code for carrying out operations of the present invention may be written in any combination of one or more programming languages, including an object-oriented programming language such as Java, Smalltalk, C++ or the like and conventional procedural programming languages, such as the C programming language or similar programming languages.

It will be understood that the algorithms described herein, can be implemented by computer program instructions. These computer program instructions may be provided to a processor of a general-purpose computer, special purpose computer, or other programmable data processing apparatus to produce a machine, such that the instructions, which execute via the processor of the computer (e.g., computer processor 32 and/or 45) or other programmable data processing apparatus, create means for implementing the functions/acts specified in the algorithms described in the present application. These computer program instructions may also be stored in a computer-readable medium (e.g., a non-transitory computer-readable medium) that can direct a computer or other programmable data processing apparatus to function in a particular manner, such that the instructions stored in the computer-readable medium produce an article of manufacture including instruction means which implement the function/act specified in the algorithms. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the algorithms described in the present application.

Computer processor 32 and/or computer processor 45 is typically a hardware device programmed with computer program instructions to produce a special purpose computer. For example, when programmed to perform the algorithms described with reference to the figures, computer processor 32 and/or 45 typically acts as a special purpose image-generating computer processor. Typically, the operations described herein that are performed by computer processor 32 and/or 45 transform the physical state of a memory, which is a real physical article, to have a different magnetic polarity, electrical charge, or the like depending on the technology of the memory that is used. For some applications, operations that are described as being performed by a computer processor are performed by a plurality of computer processors in combination with each other.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1.-40. (canceled)

41. A method for use with a tool configured to be placed within a portion of a body of a patient, the method comprising:
tracking, using a first tracking device from a first line of sight, at least a portion of the tool and a patient marker that is configured to be placed upon the body of the patient, wherein the first tracking device is disposed upon a first head-mounted device that is configured to be worn by a first person, the first head-mounted device including a first head-mounted display;
tracking, using a second tracking device from a second line of sight, at least the portion of the tool and the patient marker, wherein the second tracking device is disposed upon a second head-mounted device that is configured to be worn by a second person; and
using at least one computer processor, generating an augmented reality image upon the first head-mounted display, based upon data received from the first tracking device in combination with data received from the second tracking device, the augmented reality image including (a) a virtual image of the tool and anatomy of the patient, overlaid upon (b) the patient's body.

42. The method of claim 41, wherein the tracking, using the first tracking device from the first line of sight, at least the portion of the tool comprises tracking a tool marker.

43. The method of claim 42, wherein the tracking, using the second tracking device from the second line of sight, at least the portion of the tool comprises tracking the tool marker.

44. The method of claim 41, wherein the second head-mounted device includes a second head-mounted display, and the method further comprises generating a further augmented reality image upon the second head-mounted display.

45. The method of claim 41, wherein the first tracking device comprises a first camera configured to image at least the portion of the tool and the patient marker.

46. The method of claim 45, wherein the second tracking device comprises a second camera configured to image at least the portion of the tool and the patient marker.

47. The method of claim 41, wherein generating the augmented reality image upon the first head-mounted display comprises:
determining a position of the tool with respect to the patient's anatomy using data received from the first tracking device in combination with data received from the second tracking device;
generating the virtual image of the tool and anatomy of the patient upon the first head-mounted display, based upon the determined position of the tool with respect to the patient's anatomy;
determining a position of the patient's body with respect to the first head-mounted device based upon data received from the first tracking device; and
overlaying the virtual image upon the patient's body, based upon the determined position of the patient's body with respect to the first head-mounted device.

48. The method of claim 47, wherein determining the position of the tool with respect to the patient's anatomy using data received from the first tracking device in combination with data received from the second tracking device comprises determining an average position of the tool with respect to the patient's anatomy.

49. The method of claim 47, wherein determining the position of the patient's body with respect to the first head-mounted device based upon data received from the first tracking device comprises analyzing one or more images of the patient marker created by a camera of the first tracking device.

50. The method of claim 49, wherein the one or more images of the patient marker comprise data that is sufficient to determine the position of the patient's body with respect to the first head-mounted device without requiring use of triangulation techniques.

51. A system for use with a tool configured to be placed within a portion of a body of a patient, the system comprising:
a patient marker configured to be placed upon the patient's body;
a first head-mounted device configured to be worn by a first person, the first head-mounted device comprising a first head-mounted display, and a first tracking device that is configured to track at least a portion of the tool and the patient marker from a first line of sight;
a second head-mounted device configured to be worn by a second person, the second head-mounted device comprising a second tracking device that is configured to track at least the portion of the tool and the patient marker from a second line of sight; and
at least one computer processor configured to generate an augmented reality image upon the first head-mounted display, based upon data received from the first tracking device in combination with data received from the second tracking device, the augmented reality image including (a) a virtual image of the tool and anatomy of the patient, overlaid upon (b) the patient's body.

52. The system of claim 51, wherein the tool includes a tool marker, and wherein the first tracking device is configured to track the portion of the tool by tracking the tool marker.

53. The system of claim 52, wherein the second tracking device is configured to track the portion of the tool by tracking the tool marker.

54. The system of claim 51, wherein the second head-mounted device comprises a second head-mounted display, and wherein the at least one computer processor is further configured to generate a further augmented reality image upon the second head-mounted display.

55. The system of claim 51, wherein the first tracking device comprises a first camera.

56. The system of claim 55, wherein the second tracking device comprises a second camera.

57. The system of claim 51, wherein the at least one computer processor is configured to generate the augmented reality image upon the first head-mounted display by:
determining a position of the tool with respect to the patient's anatomy using data received from the first tracking device in combination with data received from the second tracking device;
generating the virtual image of the tool and anatomy of the patient upon the first head-mounted display, based upon the determined position of the tool with respect to the patient's anatomy;
determining a position of the patient's body with respect to the first head-mounted device based upon data received from the first tracking device; and
overlaying the virtual image upon the patient's body, based upon the determined position of the patient's body with respect to the first head-mounted device.

58. The system of claim 57, wherein determining the position of the tool with respect to the patient's anatomy using data received from the first tracking device in combination with data received from the second tracking device comprises determining an average position of the tool with respect to the patient's anatomy.

59. The system of claim 57, wherein determining the position of the patient's body with respect to the first head-mounted device based upon data received from the first tracking device comprises analyzing one or more images of the patient marker created by a camera of the first tracking device.

60. The system of claim 59, wherein the one or more images of the patient marker comprise data that is sufficient to determine the position of the patient's body with respect to the first head-mounted device without requiring use of triangulation techniques.
